(19) United States
(12) Patent Application Publication
(10) Pub. No.: US 2006/0241672 A1
(43) Pub. Date: Oct. 26, 2006

(54) Infra-epidermic subcision device for blunt dissection of sub-epidermic tissues

(76) Inventors: Filiberto P. Zadini, North Hills, CA (US); Giorgio C. Zadini, Camarillo, CA (US)

Correspondence Address: FILIBERTO P. ZADINI, 2237 HILLTOP LANE, CAMARILLO, CA, 93012, US

(21) Appl. No.: 11/112,996

(22) Filed: Apr. 21, 2005

(51) Int. Cl. A61M 29/00

(52) U.S. Cl. 606/192

(57) ABSTRACT

A dermatological device for infra-epidermic subcision via blunt dissection of fibrous bands of the edematous-fibrosclerotic panniculopathy, including a needle and a subepidermic skin layer anchoring member such as a balloon in flow communication with the needle. The fibrous bands are dissected bluntly by the balloon via dissection parallel to the surface of the skin as result of radial balloon expansion and via dissection perpendicular to the surface of the skin caused by traction on the skin induced by traction exerted by the operator upon the device.

### FIELD OF THE INVENTION

This invention relates to medical apparatuses and methods for treatment of dermatological conditions, specifically for treatment of the edematous-fibrosclerotic panniculopathy, commonly known as cellulite.

### BACKGROUND

Description of the Prior Art

Numerous treatments have been devised for the dermatological condition edematous-fibrosclerotic panniculopathy, commonly known as cellulite.

Some of these treatments have a scientific base, some have a pseudo-scientific, empiric base.

The edematous-fibrosclerotic panniculopathy commonly named cellulite, a non medical term coined in Europe, is a disorder of the skin and subcutaneous tissue. The edematous-fibrosclerotic panniculopathy is due to the formation of an abnormal fibrous network in the hypoderm. The abnormal fibrous network encapsulates conglomerates of fat cells causing a subcutaneous architectural disruption which results in a dimples and nodules appearance of the skin, known as orange peel skin. Strands of fibrous tissue connect the skin to deeper tissue layers and also separate compartments that contain conglomerates of fat cells. Cellulite affects more commonly the hips, thighs, glutei, abdominal wall and upper arms. Women are commonly more affected than men. Researchers agree that most of cellulite “cures” have been ineffective. Recent researches have confirmed that cellulite is product of faulty anatomy, genes and hormones.

Anticellulite products with unsubstantiated claims of successful treatment of the condition include creams and gels, brushes, rollers, body wraps toning lotions, electrical stimulation devices, vibrating machines, inflatable hip-high pressurized boots, hormone or enzymes injections and many others.

More recently, radio frequency and laser devices, cold-laser massage devices, combined radio frequency/infrared devices, fat melting injections, targeted liposuction, tissue fillers have been used for the treatment of cellulite with minimal or marginal success, eventually with only transitory improvements.

A more recently devised surgical procedure called skin subcision has shown some promising results. The procedure consists of cutting the cellulitic fibrous bands, the tethers which cause the depression in the skin with a special needle having surgical scalpel-like tip. The dimples, freed from their fibrous attachments, pop up and the skin is able to regain the even, pre-cellulitic aspect. Regretfully, the procedure is not void of complications. Pain, bruises, hemosiderosis have been associated with the procedure as reported in the International Journal of Dermatology, Volume 39 Issue 7, Page 539, July 2000.

### BRIEF SUMMARY OF THE INVENTION

With the present invention, applicants propose a simple medical-surgical device having the scientific prerequisites of being capable of detaching the fibrous attachments that connect the skin to the deeper layers and cause the typical dimples of a cellulitic skin, via blunt dissection, rather than via sharp dissection as currently in use. Detachment of such fibrous attachments resolves the skin dimples, restituting normal appearance to the skin, minimizing complications more likely to develop with sharp dissection.

The device is composed of a needle having an expandable balloon in proximity of the tip, connected to a syringe provided with a handle.

The operator inserts the needle into the skin, inflates the balloon, grossly shaped as a donut. The balloon once inflated has the double function of dissecting by outward radial expansion the fibrous bands network and of serving as anchoring device for skin traction purposes. The operator gently pulls up the needle acting upon the syringe handle connected to the needle carrying the expanded balloon. In doing so the operator elevates the skin, stretching it to the point of rupture the cellulitic fibrous bands which cause the dimpling of the skin.

The detachment of the fibrous bands occur by blunt dissection. It is expected that the extensible surrounding blood vessels are just stretched and not severed as in the above mentioned sharp subcision technique. Surrounding structures will be less traumatized being not sharply cut as in the sharp subcision technique. It is reasonable to say that less trauma to the tissue is expected to occur with greater patient comfort and with expectation of lesser complications.

### OBJECT OF THE PRESENT INVENTION

It is an object of the present invention to provide a simple, rapidly deployable medical device for the treatment of cellulite, the treatment being based on solid anatomic-pathological foundations.

It is an object of the present invention to provide the consumer with a simple minimally invasive effective, rapidly deployable means and method for improving cosmetic appearance of the skin affected by cellulite.

It is an object of the present invention to provide a safe, simple and effective apparatus and method to target and to induce mechanical lysis of the fibrous bands which are at the core of the formation and persistence of the cellulite in body areas of patient's concern.

It is an object of the present invention to provide the operator with an alternative improved apparatus and method of an already proven effective method of cellulite treatment i.e. skin subcision the dissection of the cellulitic fibrous bands. The proposed device dissects the fibrous tissue by blunt, not sharp, dissection, causing less trauma, less bleeding, ultimately less inflammatory reaction in the subcutaneous tissue.

### DRAWING FIGURES

FIG. 1 is a side view of device with the balloon deflated at rest prior to use.

FIG. 2 is a side view of the device with the balloon inflated.

FIG. 3 is as side view of the same device with a larger balloon fully inflated.

FIG. 4 is across sectional view of a detail of the device of FIG. 2 to 3 specifically the inflatable member or balloon or bluntly dissecting member or anchoring member inflated.

FIG. 5 shows a detail of the device specifically the locking mechanism for the plunger of device prior to actuation of the locking mechanism.

FIG. 6 shows a cross sectional view of the skin of a patient with the device in action with the balloon deployed pulled upward by the operator resulting in blunt dissection/disruption of the cellulitic fibrous bands at the skin attachment and or at the deeper layer attachment.

### DETAILED DESCRIPTION OF THE INVENTION

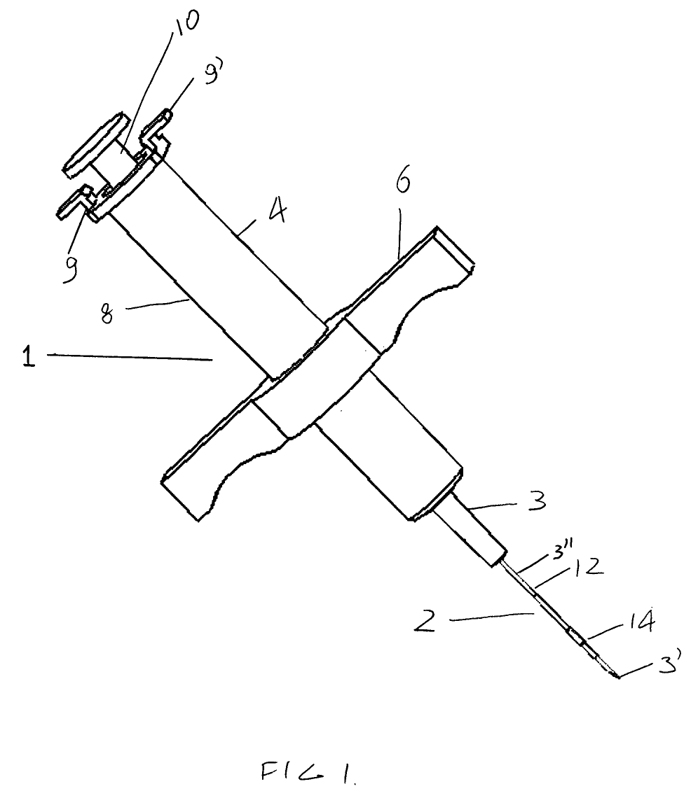

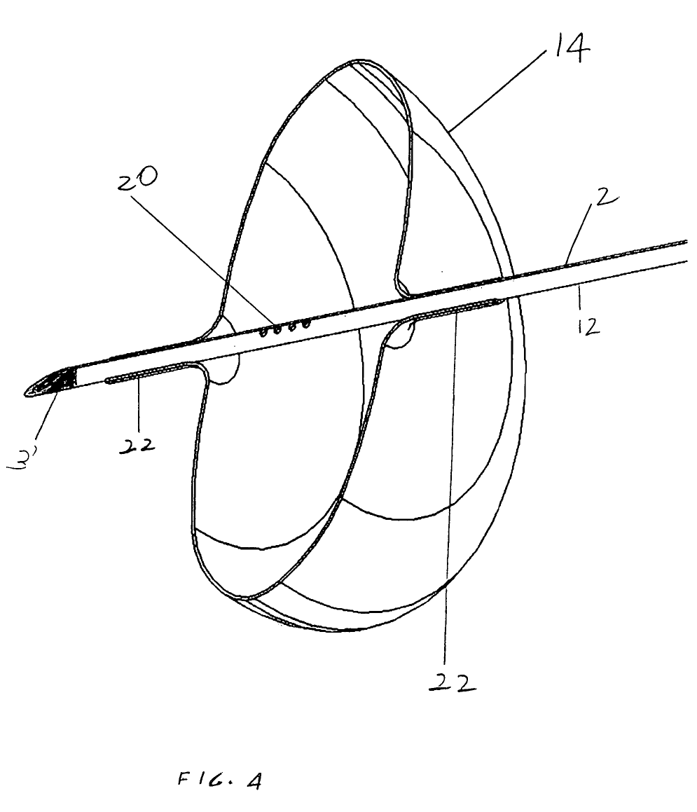

As shown in FIG. 1, Infra-epidermic Subcision Device for Blunt Dissection of Sub-epidermic Tissues or Skin blunt Dissector/Elevator 1 consists of hollow hypodermic needle or skin penetrating means 2 sufficiently rigid to allow skin perforation connected to and in flow communication with syringe or inflating means 4. Needle 2 is in tight sealing connection with syringe 4 via detachable hub 3. Needle tip segment 3′ of needle 2 is imperforated as better shown in FIG. 4, while the remaining segment 3″ of the needle is hollow. Syringe 4 is formed with barrel 8, slideable piston or plunger 10 and handle or handling means or traction or pulling means 6. Syringe is formed at its proximal end with plunger locking mechanism 9 formed with flanges 9′ for the release of locking mechanism 9.

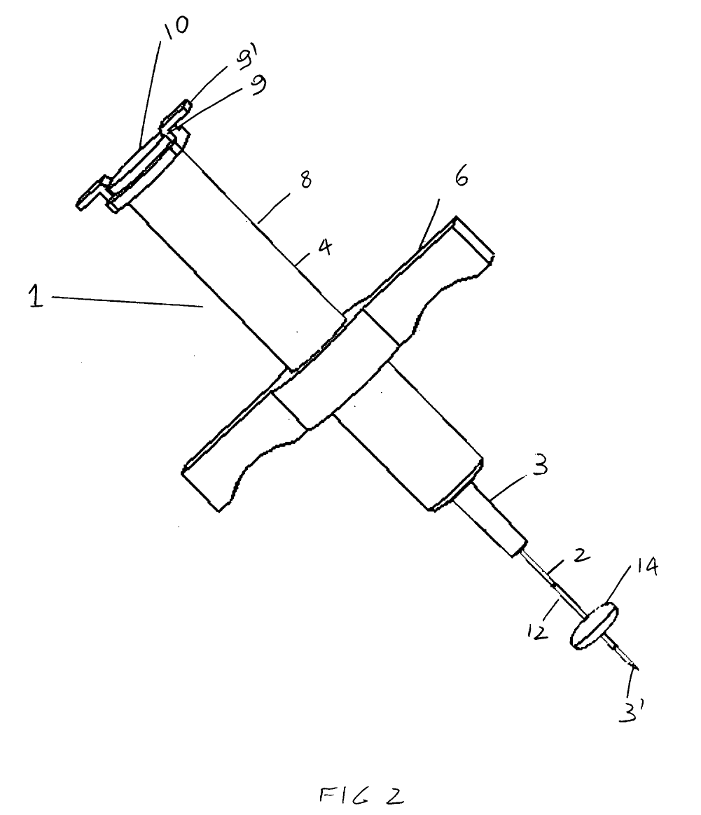

As shown in FIG. 1, balloon or expandable member or bluntly dissecting member or anchoring member 14, grossly donut shaped once inflated as shown in FIGS. 2 and 3, 4 and 6 is mounted on needle shaft 12 of needle 2.

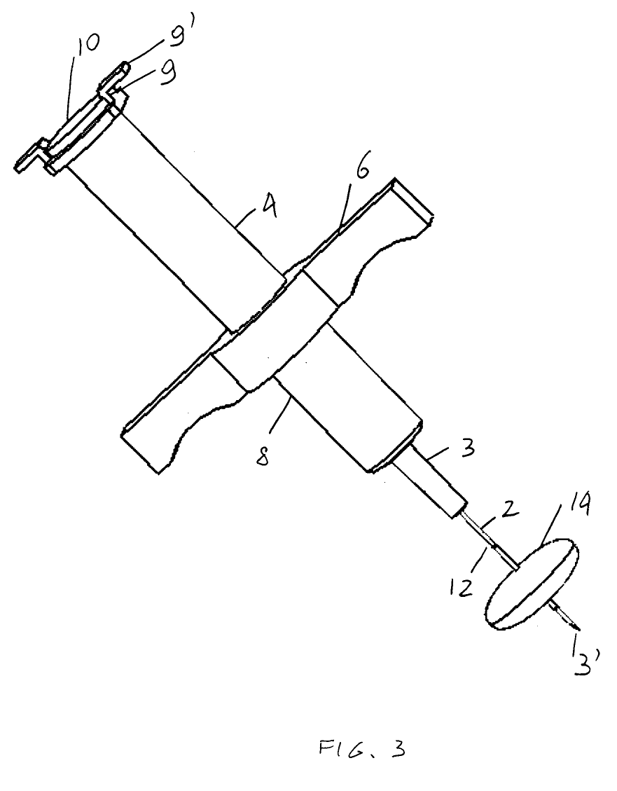

As better shown in shown in FIG. 4 which is a blown up cross sectional view of needle 2 distal segment, balloon 14, shown inflated, is in flow communication with hollow needle 2 via needle holes or needle perforations 20. Needles holes 20 are proximal to imperforated needle tip 3′. Balloon 14 of FIG. 1,2,4,6 or balloon 14′ of FIG. 3, made of extensible material up to a maximum point of expansion, is sealingly attached to needle shaft 12 via cylindrically shaped balloon extensions or sleeve 22 and 22′ as better shown in FIGS. 1 and 4.

Needle 2 can be formed with different sizes balloons allowing variable radial balloon expansions.

FIG. 3 shows device 1 with larger diameter balloon 14′ for radial-lateral blunt dissection/disruption of cellulitic fibrous bands.

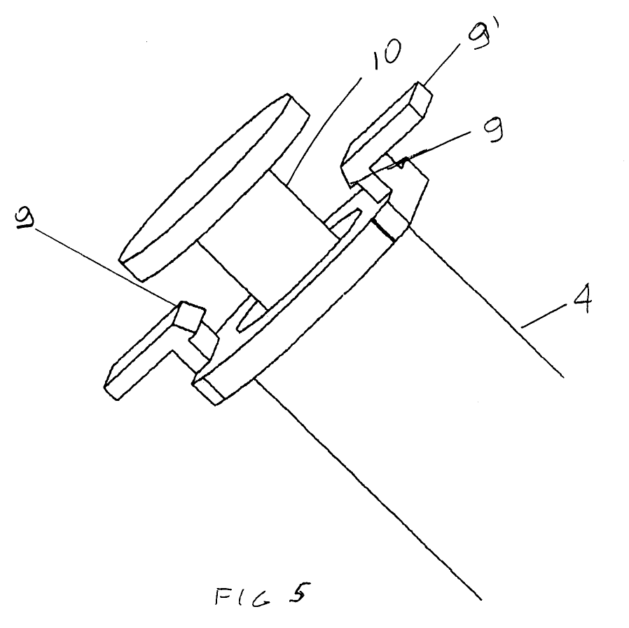

As better shown in FIG. 5, plunger locking members or mechanism 9 of plunger 10 is releasable upon pressing down on flanges 9′ which disengage locking members 9 from plunger 10.

As shown in FIG. 1, plunger 10, at rest prior to use, is withdrawn to a degree just sufficient to fully inflate balloon 14 once plunger 10 is fully downwardly displaced.

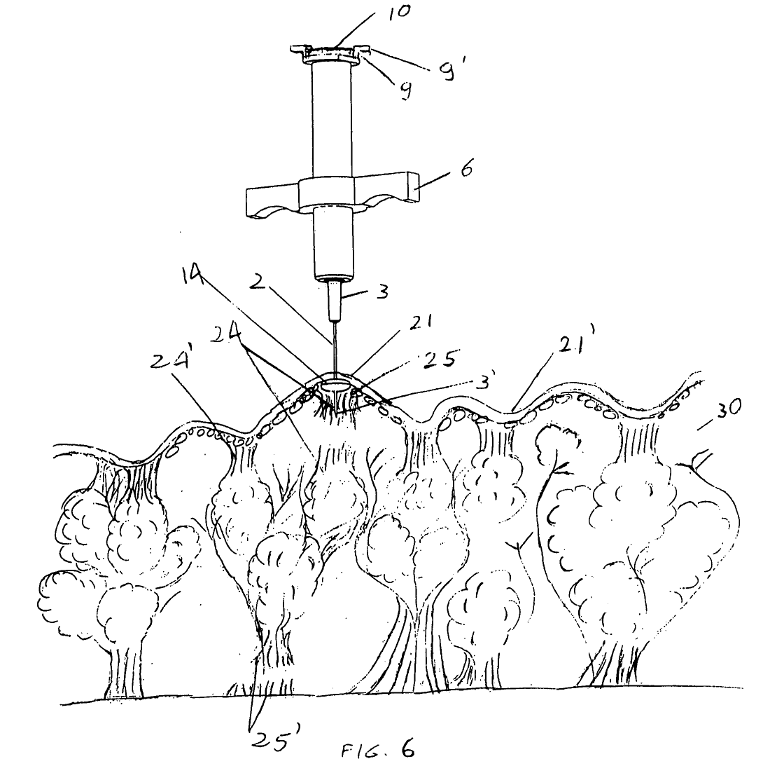

As it can be better understood from FIG. 6, which shows the device in use, the operator advances needle 2 with imperforated tip 3′ into the patient skin 30. Local anesthetic can be administered prior to skin insertion of needle tip 3′ for pain relief Needle 2 is preferably inserted in the depressed center of a skin dimple 21′ of the cellulitic skin 30. Dimple 21′ is shown before skin traction, while dimple 21 is shown in FIG. 6 during skin traction, as it will be described below. Once needle tip 3′ and distal segment of needle shaft 12 with balloon 14 is at sufficient depth underneath the epidermis, balloon 14 is inflated by the operator by advancement of plunger 10. Upon full advancement, plunger 10 is locked by locking mechanism 9 in its fully advanced position, as shown in FIG. 2, 3 and 6. Upon full advancement of plunger 10, balloon 14 inflates and expands radially-laterally. Radial-lateral expansion of balloon 14 and to a larger degree of balloon 14′ of larger diameter, will stretch cellulitic fibrous bands 24 to a point of rupture, via blunt dissection or disruption. Cellulitic fibrous bands are shown in FIG. 6 before blunt disruption 24′ and after disruption at 24. When fully expanded, balloons 14 or 14′ act as subepidermic anchoring device for skin traction. The operator pulls the device away from the skin surface via handle or traction means 6. Balloon or expandable member or bluntly dissecting member or anchoring member 14 or 14′ grossly donut shaped, subepidermically placed indeed act as anchoring member allowing elevation/traction of the skin. By elevating the skin, fibrous bands 24 are bluntly disrupted and dissected from attachments to epidermis 25 or from attachments to the deeper skin layers 25′, as shown in FIG. 6. Skin dimples 21, no longer tethered down by fibrous bands 24 and or 24′ will be free to rise by natural resiliency to the level of the surrounding skin.

The operator can repeat the procedure by inserting the needle into each cellulitic skin dimple 21′. By operating the device as described, the operator can eliminate, one by one, every skin dimple, restituting normal appearance to the skin.

What we claim is:

1. A device for infra-epidermic blunt dissection of a skin of a patient comprising:
a rigid needle having a hollow segment and an imperforated tip segment,
an inflatable member firmly connected to said needle, said inflatable member being in flow communication with said hollow segment of said needle, and
inflating means in flow communication with said hollow segment of said needle, causing expansion of said inflatable member upon placement of said inflatable member beneath a superficial layer of the skin, said expansion causing blunt dissection of tissues laying beneath said superficial layer of the skin.

2. The device of claim 1 further comprising handle means to allow manual displacement of said inflatable member away from the skin surface and consequent traction of a segment of said superficial layer of skin being engaged by said expanded member, said traction of said skin segment resulting with blunt dissection of said tissues from said superficial layer of skin.

3. A device for infra-epidermic blunt dissection of a skin of a patient comprising:
a rigid needle having a shaft segment and a tip segment for skin penetration,
an expandable member firmly connected to said needle,
expanding means to enable expansion of said expandable member, upon placement of said expandable member beneath a superficial layer of the skin, said expansion causing blunt dissection of tissues laying beneath said superficial layer of the skin.

4. The device of claim 1 further comprising handle means to allow manual displacement of said expandable member away from the skin surface and consequent traction of a segment of said superficial layer of skin being engaged by said expanded member, said traction of said skin segment resulting with blunt dissection of said tissues from said superficial layer of skin.

5. A device for skin elevation of a patient comprising:
a skin penetrating member and
anchoring means connected to said skin penetrating means said anchoring means being placeable in a contracted state under a superficial layer of the skin, said anchoring means being deployable under the skin after skin penetration by said skin penetrating means, allowing, after being deployed, traction upon the skin, said skin traction resulting in blunt dissection of tissues under the superficial layer of the skin.
